(19) United States
(12) Patent Application Publication
(10) Pub. No.: US 2006/0205977 A1
(43) Pub. Date: Sep. 14, 2006

(54) Processes for producing terephthalic acid

(76) Inventors: Charles Edwan Sumner JR., Kingsport, TN (US); Robert Thomas Hembre, Johnson City, TN (US); David Lange, Irmo, SC (US); Gino Georges Lavoie, Kingsport, TN (US); Brent Alan Tennant, Kingsport, TN (US); Thomas Richard Floyd, Kingsport, TN (US); Bryan Wayne Davenport, Columbia, SC (US); Daniel Burts Compton, Kingsport, TN (US); Joseph Nathaniel Bays, Kingsport, TN (US)

Correspondence Address: Michael K. Carrier;Eastman Chemical Company, P.O. Box 511, Kingsport, TN, 37662-5075, US

(21) Appl. No.: 11/214,406

(22) Filed: Aug. 29, 2005

Related U.S. Application Data
(60) Provisional application No. 60/659,715, filed on Mar. 8, 2005.

(51) Int. Cl. C07C 51/16; C07C 51/255

(52) U.S. Cl. 562/410

(57) ABSTRACT

Processes for producing terephthalic acid are disclosed, the processes comprising combining in a reaction medium p-xylene, a solvent comprising water and one or more saturated organic acids having from 2-4 carbon atoms, and an oxygen-containing gas, at a temperature from about 135° C. to about 165° C., in the presence of a catalyst composition comprising cobalt atoms and manganese atoms, with bromine atoms provided as a promoter. The amount of cobalt used may be from about 1,800 ppm to about 6,000 ppm, with respect to the total weight of the liquid in the reaction medium, and the weight ratio of cobalt to manganese may be from about 40 to about 400. The processes according to the invention produce terephthalic acid as a precipitated reaction product, with typically no more than about 10 ppm 2,6-dicarboxyfluorenone produced, with respect to the weight of the terephthalic acid produced, or no more than about 20 ppm 2,6-dicarboxyfluorenone, with respect to the total weight of the reaction medium, per batch or upon one pass through a reactor. The terephthalic acid so produced may be further purified by one or more additional oxidation reactions, without the need for expensive hydrogenation purification processes.

### CROSS-REFERENCE TO RELATED APPLICATIONS

This application claims priority to U.S. Provisional Application Ser. No. 60/659,715, filed Mar. 8, 2005 the disclosure of which is incorporated herein by reference in its entirety.

### FIELD OF THE INVENTION

This invention pertains to improved processes for the production of terephthalic acid by the liquid-phase oxidation of para-xylene, the processes resulting in reduced formation of impurities that cause color in subsequent polymerization products, while maintaining acceptable conversion and oxidative solvent loss.

### BACKGROUND OF THE INVENTION

Aromatic dicarboxylic acids such as terephthalic acid and isophthalic acid are used to produce a variety of polyester products, important examples of which are poly(ethylene terephthalate) and its copolymers. These aromatic dicarboxylic acids may be synthesized by the catalytic oxidation of the corresponding dialkyl aromatic compound. For example, terephthalic acid (TPA) and isophthalic acid (IPA) may be produced by the liquid phase oxidation of p-xylene and m-xylene, respectively.

These processes typically comprise feeding one or more dialkyl aromatic hydrocarbons, fresh and/or recycled solvent or reaction medium, and catalyst components to a reactor to which a molecular oxygen-containing gas also is fed, typically near the bottom of the reactor. Conventional liquid-phase oxidation reactors are equipped with agitation means for mixing the multi-phase reaction medium. Agitation of the reaction medium is supplied in an effort to promote dissolution of molecular oxygen into the liquid phase of the reaction medium, and to facilitate contact between the dissolved oxygen and the diakyl aromatic hydrocarbon in the reaction medium. This agitation is frequently provided by mechanical agitation means in vessels such as, for example, continuous stirred tank reactors (CSTRs). Bubble column reactors provide an attractive alternative to CSTRs and other mechanically agitated oxidation reactors. The bubble column reactors used typically have a relatively high height to diameter ratio. The oxygen-containing process gas rising through the liquid contents of the reactor results in agitation of the reaction mixture. Alternatively, continuous stirred tank reactors may be used, typically having a lower height to diameter ratio than the bubble columns.

Regardless of reactor design, the aromatic dicarboxylic acid produced is typically removed continuously through an exit port as a slurry. Process gas containing excess oxygen, along with solvent decomposition products, may be removed through an upper exit port typically located at or near the top of the reactor. The heat of reaction may also be removed through the upper exit port by vaporization of the process solvent and the water generated by the reaction.

Thus, in one example of such a process, p-xylene is oxidized to produce terephthalic acid. The p-xylene may be continuously or batch-wise oxidized in the primary oxidation reactor in the liquid phase, in the presence of an oxygen-containing gas such as air. In such a process, p-xylene, an oxidation catalyst composition, a molecular source of oxygen, and a solvent such as aqueous acetic acid are combined as a reaction medium in the reactor to produce a crude terephthalic acid (CTA) reaction product. Suitable oxidation catalyst compositions include a cobalt compound and a manganese compound, usually in combination with a promoter such as a bromine compound. See, for example, U.S. Pat. Nos. 2,833,816, 3,089,906, and 4,314,073, the disclosures of which are incorporated herein by reference. The process conditions are highly corrosive due to the acetic acid and bromine, and titanium is typically used in the process equipment. See, for example, U.S. Pat. No. 3,012,038, incorporated herein by reference. Acetaldehyde may be used as a promoter in place of bromine, in which case titanium materials need not be used. Acetaldehyde is also useful as an initiator. Because these liquid-phase oxidations of dialkyl aromatic compounds are highly exothermic reactions, they are commonly carried out in a vented, columnar reaction vessel.

The resulting crude terephthalic acid mixture (CTA) is not very soluble in the acetic acid solvent under the reaction conditions, and precipitates from the solvent to form a suspension that includes terephthalic acid solids, a solvent acting as the suspending medium for the solids and containing a small amount of dissolved terephthalic acid; catalyst components; unreacted p-xylene; incompletely oxidized intermediate oxidation products such as para-tolualdehyde (p-TAl), para-toluic acid (p-TA), and 4-carboxybenzaldehyde (4-CBA); and organic impurities that are known to cause discoloration, and especially fluorenones. The crude terephthalic acid composition is discharged from the oxidation zone and subjected to any of several mother liquor exchange, separation, purification, or recovery methods, with the recovered solvent and catalyst composition being recycled directly back to the oxidation reaction or after processing, such as by catalyst recovery or solvent purification. It is desirable to reduce the amount of incompletely oxidized intermediates and colored impurities, to reduce the subsequent purification requirements. This is especially the case with colored compounds such as fluorenones which, when present in significant quantities, require hydrogenation in a subsequent purification process. Processes that avoid the formation of significant amounts of these colored compounds allow purification by a secondary oxidation step, avoiding the expense of a separate hydrogenation step.

Other by-products of the liquid phase oxidation which are partially or completely removed from the reaction mixture in the oxidation reactor are removed as off-gases. These off-gases include water, solvent, unreacted oxygen and other unreacted gases found in the source of the molecular oxygen gas such as nitrogen and carbon dioxide, and additional amounts of carbon dioxide and carbon monoxide produced by the catalytic decomposition of the solvent and other oxidizable compounds under the oxidation conditions. These off-gases also include gaseous bromine compounds, such as methyl bromide, that represent a loss of the bromine promoter from the reaction mixture.

Although it is desirable to recover and recycle as much solvent as possible, the solvent is oxidatively decomposed to some extent into its constituent gaseous products, carbon dioxide and carbon monoxide, requiring a fresh source of make-up solvent. This oxidative decomposition is often referred to in the industry as solvent burn or acid burn, and is generally believed to be primarily responsible for the formation of carbon oxides, although it is possible that at least a portion of the carbon oxides produced is the result of oxidative decomposition of the dialkyl aromatics or intermediate reaction products. Controlling or reducing formation of carbon oxides would significantly lower the operating costs of the oxidation process, by allowing a greater amount of solvent to be recovered and recycled back to the oxidation zone, and also by reducing yield loss from the oxidative decomposition of the aromatic reactants. However, a reduction in carbon oxides formation should not come at the expense of significantly reduced yield or conversion, or an increase in the amount of incomplete oxidation products in the crude mixture, and if possible, it would be desirable to simultaneously reduce carbon oxides formation and increase the conversion. Typically, however, increased conversion is accompanied by an increase in carbon oxides formation.

Thus, the amount of carbon oxides formed, including both carbon monoxide and carbon dioxide, can be used as a means to estimate acid burn. The amount of methyl acetate formed may also be used to estimate acid burn. Without wishing to be bound by any theory, the methyl acetate is believed to arise from the reaction of methanol with acetic acid, the methanol being produced from the reaction of methyl radicals with oxygen. Although some of the carbon oxides and methyl acetate formed may arise instead from sources other than acetic acid, controlling or reducing carbon oxides formation, as well as the formation of methyl acetate, would significantly lower the operating costs of the oxidation process, by allowing a greater amount of solvent to be recovered and recycled back to the oxidation zone, thus lowering the amount of fresh make-up feed required, or by avoiding a loss in yield caused by oxidation of the reactant or one or more of the aromatic intermediates. Again, this reduction in carbon oxides formation should not come at the expense of significantly reduced yield or conversion, or an increase in the amount of incomplete oxidation products, such as 4-CBA, in the crude mixture, and if possible, it would be desirable to simultaneously control the solvent burn and reduce the amount of 4-CBA and colored compounds generated in the crude oxidation mixture.

As noted, in addition to acid burn and loss of bromine promoter, the generation of colored byproducts during the oxidation is also undesirable. Carboxyfluorenone isomers, such as 2,6-dicarboxyfluorenone, have been identified as a major source of color in CTA. In conventional processes, these highly-colored carboxyfluorenone compounds, when present in significant amounts, must be removed or converted to colorless carboxyfluorene compounds, such as by hydrogenation. It would clearly be an advance in the art to avoid formation of significant amounts of these colored compounds, so as to avoid the need for subsequent hydrogenation processes.

It is generally understood that increased reaction temperatures and reaction rates result in increased formation of color bodies that are afterward difficult to remove from the product. The present applicants have discovered that, although the amount of fluorenone derivatives produced during these oxidations typically increases with increasing reaction temperature, lower reaction temperatures may also result in increased formation of color bodies, due to lower rates of conversion to the ultimate product. In the processes according to the invention, moderate temperatures may therefore be used, in combination with relatively high amounts of cobalt in the catalyst compositions with respect to the amount of manganese used, to obtain a product having low levels of color bodies, with acceptable conversion and carbon oxides formation.

U.S. Pat. No. 3,089,906 describes a process by which mixed xylenes may be oxidized via a liquid phase oxidation using a catalyst system comprising a heavy metal oxidation catalyst and a source of bromine.

U.S. Pat. No. 4,314,073 describes a process in which p-xylene is oxidized to terephthalic acid using a Co/Mn/Br catalyst in acetic acid, and the crude slurry product is then purified by treating with molecular oxygen and diluting with fresh acetic acid before separation of the terephthalic acid and mother liquor. p-Carboxybenzaldehyde is said to be the most serious impurity in the terephthalic acid, and the patent is therefore directed to an improved purification step. Although the patent suggests a wide range of temperatures, pressures, and catalyst concentrations, the disclosure neither teaches nor suggests that moderate temperatures may be used in combination with relatively high ratios of cobalt to manganese to achieve acceptable conversion with reduced color body formation.

U.S. Pat. No. 4,827,025 describes a process for producing aromatic carboxylic acid in a continuous manner by oxidizing an alkyl aromatic compound in the liquid phase with an oxygen-containing gas in the presence of heavy metal compound(s) and/or bromine-containing compound. According to the process described, a part of the reaction gas delivered from the reactor and freed from the condensing components is recirculated by returning it to the reactor at a position within the gas region. The process is said to eliminate the troubles due to foaming occurring on the liquid surface in the reactor. Although the disclosure suggests a wide range of temperatures and catalyst concentrations, the temperatures exemplified are relatively high, and the disclosure neither teaches nor suggests that moderate temperatures may be used in combination with relatively high ratios of cobalt to manganese to achieve acceptable conversion with reduced color body formation.

EP 0 673 910 A1 suggests, in liquid phase oxidations of alkylbenzenes, the use of an oxygen-containing gas having an oxygen content higher than that of air. The document exemplifies relatively high reaction temperatures with relatively low cobalt amounts and cobalt to manganese ratios.

U.S. Pat. No. 5,763,648 describes a process for producing an aromatic carboxylic acid, in which an alkylaromatic hydrocarbon is oxidized with a molecular oxygen-containing gas in the liquid phase in an acetic acid solvent in the presence of catalyst components comprising cobalt, manganese and bromine. According to the process described, the reaction temperature is from 140° C. to 180° C.; the cobalt component is in an amount of from 400 to 3,000 ppm by weight of the acetic acid solvent; and a part of the oxidation exhaust gas withdrawn from the reactor is recycled back to the liquid phase, the reaction pressure being adjusted to a level which is higher than the pressure for a case where air is used as the molecular oxygen-containing gas and no recycling of the oxidation exhaust gas is carried out. This disclosure likewise neither teaches nor suggests the processes of the present invention in which moderate temperatures may be used in combination with relatively high ratios of cobalt to manganese to achieve acceptable conversion with reduced color body formation.

Likewise, U.S. Pat. Nos. 2,962,361, 3,089,906, 3,970,696, 4,159,307, 4,327,226, 5,679,847, 5,756,833, U.S. Pat. Publn. No. 2002/0193630 A1, U.S. Pat. Publ. No. 2002/0183546, JP 1997278709A, and GB1 389 478 suggest the use of catalyst systems that include cobalt, manganese, and bromine, but none teach or suggest the processes of the present invention in which moderate temperatures may be used in combination with relatively high ratios of cobalt to manganese to achieve acceptable conversion with reduced color body formation.

There remains a need in the art for aromatic oxidation processes that achieve acceptable yields and conversion, while controlling oxidative solvent loss and minimizing formation of colored compounds that are difficult to remove from the product mixture. These and additional advantages are obtained by the present invention, as further described below.

### SUMMARY OF THE INVENTION

The invention relates to processes for producing terephthalic acid, the processes comprising combining in a reaction medium p-xylene, a solvent comprising water and one or more saturated organic acids having from 2-4 carbon atoms, and an oxygen-containing gas, at a temperature from about 135° C. to about 165° C., in the presence of a catalyst composition comprising cobalt atoms and manganese atoms, with bromine atoms provided as a promoter.

According to the invention, the cobalt atoms may be present, for example, in an amount from about 1,800 ppm to about 6,000 ppm, with respect to the total weight of the liquid in the reaction medium, and the weight ratio of cobalt to manganese is from about 40 to about 400. The processes according to the invention produce terephthalic acid as a precipitated reaction product, with typically no more than about 10 ppm 2,6-dicarboxyfluorenone (2,6-DCF) produced per batch or per pass through a reactor, with respect to the weight of the terephthalic acid produced, or no more than about 20 ppm 2,6-dicarboxyfluorenone produced per batch or per pass through a reactor, based on the total weight of the reaction mixture. The terephthalic acid mixture so produced may be further purified by one or more additional oxidation reactions, without the need for subsequent hydrogenation purification processes.

### BRIEF DESCRIPTION OF THE DRAWINGS

FIG. 1 illustrates a process flow of crude terephthalic acid streams and the overhead of an oxidation unit.

### DETAILED DESCRIPTION OF THE INVENTION

The present invention may be understood more readily by reference to the following detailed description of the invention, including the appended figure, and to the examples provided. It is to be understood that the terminology used is for the purpose of describing particular embodiments only and is not intended to be limiting.

As used in the specification and the claims, the singular forms “a,” “an,” and “the” include plural referents unless the context clearly dictates otherwise.

It is to be understood that the words “comprising” and “containing” are open ended and may include any number and type of unstated steps, processes, or ingredients. The description of method steps does not preclude intervening steps and is not restricted to carrying out the steps in a particular order unless otherwise stated. Numerical ranges include each integer and all fractions thereof between the end points of the stated range.

Unless otherwise indicated, the weight amount of catalyst is based in each instance on the total weight of the liquid in the reaction medium, without regard to the amount of precipitated product in the reaction medium, the amount of which may change during the course of the reaction, especially in those cases in which the process is carried out as a batch or semi-batch process. The defined weight amounts may be determined by removal of a portion of the reaction medium either during or after the reaction, since the amount present in the reaction mixture may differ somewhat from the concentration of catalyst as initially provided to the reaction mixture, due to evaporation, solvent burn, etc.

Thus, in various embodiments, p-xylene is oxidized in an aqueous aliphatic solvent, such as aqueous acetic acid, with oxygen-containing gas, in the presence of a catalyst system comprising cobalt atoms and manganese atoms, with bromine atoms provided as a promoter. The processes may be carried out at relatively low temperatures, for example from about 135° to about 165° C., or from 140° C. to 165° C., or from 140° to 160° C., with the cobalt atoms being present in an amount of at least about 1,800 ppm, or at least about 1,900 ppm, or at least 2,000 ppm, or at least 2,500 ppm, or from about 1,800 to about 6,000 ppm, or from about 1,900 to about 6,000 ppm, or from 2,000 to 5,000 ppm, each with respect to the total weight of the liquid in the reaction medium. The processes according to the invention carried out at relatively low reaction temperatures obtain a suitable yield and conversion, without unreasonably high carbon oxides formation. The processes according to the invention produce terephthalic acid as a reaction product, with typically no more than about 10 ppm 2,6-dicarboxyfluorenone produced, expressed with respect to the weight of the terephthalic acid produced, or no more than about 8 ppm, or no more than about 6 ppm, or from 0.4 to 10 ppm, or from 0.5 to 6 ppm, in each case expressed with respect to the weight of the terephthalic acid produced. As an alternative measure of 2,6-DCF content, typically no more than about 20 ppm 2,6-dicarboxyfluorenone is produced, based on the total weight of the reaction mixture, or no more than about 18 ppm, or no more than about 15 ppm, or from 1 ppm to 20 ppm, or from 2 ppm to 18 ppm, or from 3 ppm to 15 ppm, in each case based on the total weight of the reaction mixture.

The processes according to the invention thus provide low levels of 2,6-DCF while keeping the quantities of CO and CO₂ produced in the course of the reaction (believed to be a good indicator of the extent of acid burn), within an acceptable range. Such a decrease in levels of impurities generated reduces significantly the extent of purification of the terephthalic acid required.

According to the invention, the resulting terephthalic acid product mixture, typically containing no more than about 10 ppm 2,6-DCF, with respect to the weight of the terephthalic acid produced, may be subjected to one or more additional oxidation purification processes, without the need for expensive hydrogenation processes.

The levels of 2,6-DCF produced over the course of the reaction, during batch processes or continuous processes of relatively short duration, and especially those using virgin feeds of p-xylene, catalyst, and solvent, will approximate those found in the reaction medium at any given time. However, when the processes according to the invention are carried out continuously, for example with recycled filtrate, the levels of 2,6-DCF in the product mixture at any given time may be higher than those values given per batch or based on a single pass through the reactor, while remaining consistent with the proviso that no more than about 10 ppm 2,6-dicarboxyfluorenone, expressed with respect to the weight of the terephthalic acid produced, is formed per pass through the reactor. This would be the case, for example, when a partially purified catalyst recycle stream containing 2,6-dicarboxyfluorenone is fed back to the reactor, or when one or more intermediates responsible for the formation of 2,6-DCF is fed back through the reactor during the course of the reaction.

For example, feeding 2-monocarboxyfluorenone to the reaction medium might result, after methylation and subsequent further oxidation, in the formation of 2,6-DCF. Similarly, feeding back 2,5,4′-tricarboxybiphenyl may increase the concentration of 2,6-DCF in the slurry above that seen with a batch or one-pass process. 2,6-DCF might also be generated by the reaction of a m-xylyl radical arising from the presence of m-xylene in the reactant feed, with isophthalic acid, which could be found in high concentration in the recycled filtrate, after further oxidation and cyclization. In any event, the amount of 2,6-dicarboxyfluorenone produced, that is attributable to the p-xylene fed to the reaction medium, is nonetheless typically no greater than 10 ppm 2,6-dicarboxyfluorenone, expressed with respect to the weight of the terephthalic acid produced. This would, of course, also be the case when the 2,6-DCF produced is measured based on the total weight of the reaction medium, and for the same reasons.

Paradoxically, although the amount of fluorenone derivatives produced during these oxidations may increase with increasing reaction temperature, for example above about 165° C., lower reaction temperatures, for example less than about 135° C., may also result in increased formation of color bodies, due to lower conversion to the ultimate product.

Without being bound by theory, the correlation between increased temperature and increased color body formation is believed to be due to the decomposition of key intermediates during the oxidation cycle. For example, according to a proposed mechanism for the autoxidation of xylene, there should be a steady state concentration of at least two benzoyl radical species. The decarbonylation of a benzoyl radical will produce an aryl radical and an equivalent of carbon monoxide. This decarbonylation reaction is dependant upon temperature, since the enthalpy for the reaction is positive (around 20 kcal/mole). The incipient aryl radicals are thought to add to aromatic hydrocarbons with various selectivities to give a biphenyl compound, which may subsequently cyclize to form a fluorenone. Thus, a decrease in oxidation temperature would be expected to result in a slower generation of aryl radical species and a consequential decrease in fluorenone byproducts, and alternatively, an increase in reaction temperature would be expected to result in an increase in color body formation. Further discussion may be found in Fossey, J; Lefort, D.; Sorba, J; “Free Radicals in Organic Chemistry”, pp. 148-150, John Wiley & Sons, 1995; and Abramovitch, R. A.; Intra-Science Chemistry Reports, 3 (1969) 211, the disclosures of which are incorporated herein by reference.

However, a problem expected with a decrease in oxidizer temperature is a loss of conversion, and decreases in the conversion of p-TA (and consequently of 4-carboxybenzaldehyde) are associated with increases in the amounts of intermediates, such as dicarboxyfluorenone compounds, found in the product. This is thought to be due to the larger amount of soluble aromatic compounds, e.g. p-TA and hydroxymethylbenzoic acid, present in the reaction mixture at lower temperatures. The soluble aromatic compounds are thought to act as traps for aryl radicals and generate 2-substituted biphenyl derivatives that are converted to DCF under reaction conditions. However, the catalyst system according to the invention provides an increase in the rate of oxidation at moderate temperatures, so as to maintain acceptable conversion at relatively moderate temperatures.

Thus, in one embodiment, the process comprises oxidizing p-xylene in the liquid phase. The liquid phase may at any moment comprise any or all of: the feed reactants p-xylene and the oxygen-containing gas, the solvent, the catalysts, and the terephthalic acid reaction product dissolved or suspended in the reaction mixture, especially when the process is carried out as a continuous process. The products of the processes according to this embodiment include terephthalic acid solids as the predominant product (for example, at least 50 wt. % of the solids), and incomplete oxidation products which may be found in the solids, in the liquid phase, or in both. p-Xylene fed to the oxidation reactor may be purified of contaminants which may interfere with the oxidation reaction. The reactant feed may be pure or a mix of the compound isomers or lower or higher homologues, as well as some saturated alicyclic or aliphatic compounds having similar boiling points to the aromatic or fused ring compounds. However, in this embodiment, at least 80 wt. %, preferably at least 95 wt. %, at least 98 wt. %, or at least 99 wt. % of the liquid reactants is p-xylene.

According to the invention, the liquid phase oxidation processes are carried out in the presence of an aliphatic solvent. Suitable solvents are those which are solvents for p-xylene under the oxidation reaction conditions. Suitable solvents include mixtures of water and the aliphatic solvents. The preferred aliphatic solvents are aliphatic carboxylic acids, and include aqueous solutions of C₂ to C₆ monocarboxylic acids, and preferably C₂ to C₄ monocarboxylic acids, e.g., acetic acid, propionic acid, n-butyric acid, isobutyric acid, n-valeric acid, trimethylacetic acid, caprioic acid, and mixtures thereof. Preferably, the solvent is volatile under the oxidation reaction conditions to allow it to be taken as an off-gas from the oxidation reactor. It is also preferred that the solvent selected is one in which the catalyst composition is soluble under the reaction conditions.

A preferred solvent for use according to the invention is an aqueous acetic acid solution, having a concentration, for example, from about 90 to about 97 wt. % acetic acid, with respect to the weight of liquid in the reaction medium. In various embodiments, the solvent comprises a mixture of water and acetic acid which, for example, has a water content sufficient to provide at least about 3% by weight water in the reaction medium, or at least 4 wt. %, or from about 3 wt. % to about 15 wt. %, or from 3 wt. % to 11 wt. %.

The solvent and catalyst used in such processes may be recycled and reused. For example, the crude terephthalic acid composition may be discharged from the oxidation zone and subjected to a variety of mother liquor exchange, separation, purification, or recovery methods. These methods can provide recovered solvent and catalyst composition for recycling back to the oxidation zone. Thus, a portion of the solvent feed to the primary oxidation reactor may be obtained from a recycle stream obtained by displacing, for example, from about 80 to about 90% of the mother liquor taken from the crude reaction mixture stream discharged from the primary oxidation reactor with fresh, wet acetic acid. This exchange may be accomplished in any convenient apparatus but can most easily be accomplished in a centrifuging apparatus, such as one or more cyclones.

The processes according to the invention are conducted in the presence of a source of oxygen. This may be accomplished by feeding an oxygen-containing gas to the oxidation reactor to allow the gas to contact the liquid reaction mixture in the reactor. The predominately gas-phase oxidant stream introduced into the reactor comprises molecular oxygen (O₂), for example in the range from about 5 to about 100 mole percent molecular oxygen, or from about 10 to about 50 mole percent molecular oxygen, or from 15 to 25 mole percent molecular oxygen. The balance of the oxidant stream typically is comprised primarily of a gas or gases, such as nitrogen, that are inert to oxidation. Thus, the oxidant stream may comprise air containing about 21 mole percent molecular oxygen and about 78 to about 81 mole percent nitrogen. Alternatively, the oxidant stream may comprise substantially pure oxygen. In other alternatives, the amount of oxygen may be no more than about 50 mole percent, or no more than about 40 mole percent.

In the processes according to the invention, the oxidation reaction proceeds at elevated temperatures and pressures. During oxidation, the time-averaged and volume-averaged temperature of the reaction medium may be maintained, for example, in the range from about 135° C. to about 165° C., or from about 140° C. to about 165° C., or from 140° C. to 160° C. The overhead pressure above the reaction medium may, for example, be maintained in the range of from about 1 to about 20 bar gauge (barg), or from 2 to about 12 barg, or from 3 to 8 barg.

We have found according to the invention that relatively low oxidation temperatures help to reduce the extent of carbon oxides formation, other conditions being equal. The process of the invention thus is particularly well suited for oxidizing p-xylene at relatively low temperatures, as already described.

The catalyst system employed in the process of the invention comprises cobalt atoms, manganese atoms, and bromine atoms, supplied by any suitable means, such as is known in the art. In a preferred embodiment, the catalyst system consists essentially of cobalt atoms, manganese atoms, and bromine atoms. The catalyst composition is typically soluble in the solvent under reaction conditions, or it is soluble in the reactants fed to the oxidation zone. Preferably, the catalyst composition is soluble in the solvent at 40° C. and 1 atm, and is soluble in the solvent under the reaction conditions.

The cobalt atoms may be present in an amount of at least 1,800 ppm, or at least 1,900 ppm, or at least 2,000 ppm, or at least 2,500 ppm, or from about 1,800 ppm to about 6,000 ppm, or from 1,900 ppm to 6000 ppm, or from 2,000 ppm to 5000 ppm, each with respect to the weight of the liquid in the reaction medium. The cobalt atoms may be provided in ionic form as inorganic cobalt salts, such as cobalt bromide, cobalt nitrate, or cobalt chloride, or organic cobalt compounds such as cobalt salts of aliphatic or aromatic acids having 2-22 carbon atoms, including cobalt acetate, cobalt octanoate, cobalt benzoate, and cobalt naphthalate.

The weight amounts of each of cobalt, manganese, and bromine are based on the atomic weight of the atoms, whether or not the atoms are in elemental form or in ionic form. The weight of a catalyst component includes the counter-cation or anion only if the weight percentage is used in the context of the source of the atom. For example, the amount of cobalt refers to the amount of cobalt atoms, whether elemental or ionic, and not the amount of cobalt acetate. The stated concentrations of catalyst components are based on the quantity of catalyst components in the liquid portion of the reaction medium in the oxidation reactor. The catalyst component concentrations may be measured, for example, by sampling the oxidation reactor underflow.

The oxidation state of cobalt when added as a compound to the reaction mixture is not limited, and includes both the +2 and +3 oxidation states.

The source of manganese may be provided as one or more inorganic manganese salts, such as manganese borates, manganese halides, manganese nitrates, or organometallic manganese compounds such as the manganese salts of lower aliphatic carboxylic acids, including manganese acetate, and manganese salts of beta-diketonates, including manganese acetylacetonate. Manganese of the catalyst composition may be present in a concentration from about 20 to about 425 ppm, or from 20 to 300 ppm, or from 40 to 200 ppm.

The bromine component may be added as elemental bromine, in combined form, or as an anion. Suitable sources of bromine include hydrobromic acid, sodium bromide, ammonium bromide, potassium bromide, tetrabromoethane, benzyl bromide, alpha-bromo-p-toluic acid, and bromoacetic acid. Hydrogen bromide and alpha-bromo-p-toluic acid may be preferred bromine sources. Bromine may thus be present in an amount ranging from about 800 to about 4,600 ppm, based on the total liquid in the reaction medium, or from 1,200 ppm to 4,200 ppm, or from 2,000 to 4,000 ppm, each with respect to the total weight of liquid in the reaction medium.

According to the invention, the relative amounts of elements in the catalyst composition are selected so as to limit carbon oxides formation and formation of colored compounds. Thus, the weight ratio of cobalt atoms to bromine atoms may be, for example, from about 0.7 to about 3.5, or from about 0.8 to about 3.0, or from about 1.0 to about 2.5. Further, the ratio of cobalt atoms to manganese atoms may be from about 40 to about 400, or from about 40 to about 200, or from about 50 to about 150. In various other embodiments, the ratio of cobalt atoms to manganese atoms may be at least 40, or at least 45, or even 50 or greater, up to about 150, or up to about 250, or up to about 400.

Other organic or non-metallic catalyst components can be included in the catalyst composition of the invention, or the processes may be carried out in the substantial absence of additional organic or non-metallic catalysts. For example, the catalyst composition may include a source of pyridine. The pyridine component of the catalyst system may be added to a primary oxidation reactor or to post oxidation reactors. The pyridine component can be in the form of pyridine per se or in the form of a compound of pyridine.

Further, the processes according to the invention may be carried out in the presence of, or in the substantial absence of, one or more aldehydes or ketones.

Further, the processes according to the invention may be carried out in the presence of additional metal atoms, or in the substantial absence thereof, so long as the catalyst composition comprises cobalt atoms and manganese atoms, with bromine atoms provided as a promoter. Such additional metals may include, but not be limited to, sodium, potassium, copper, zirconium, hafnium, chromium, and palladium. When present, sodium may be used, for example, in an amount up to about 500 ppm, or up to about 1,000 ppm, or up to about 1,500 ppm, or from about 10 ppm to about 1,500 ppm, or from about 100 ppm to about 1,000 ppm, for example, based on the total weight of liquid in the reaction medium.

The catalyst composition can be formed by adding each source to the oxidation reactor separately, in sequence, or simultaneously, or a prepared composition may be added to the oxidation reactor, and in either case, the addition may be made as an initial batch or continuously during the course of the oxidation reaction. The catalyst composition prepared as a batch may be dissolved in the solvent to form a catalyst feed followed by adding the catalyst feed to the primary oxidation reactor. Each component, or the catalyst composition batch, can be added to the primary oxidation reactor before, during, or after addition of the solvent. In a continuous process, the catalyst components or the catalyst composition may be added simultaneous with the solvent feed, or in the solvent feed, or separately metered as required for fresh make-up.

After the initial charge of catalyst composition in a continuous process, the residual mother liquor from the primary oxidation may supply a portion of the necessary catalyst components to the primary oxidation reactor by partial displacement of the primary oxidation mother liquor with fresh solvent. The remainder can be made up with a continuous fresh feed of make-up catalyst. As already described, when the process according to the invention is carried out continuously, the levels of 2,6-DCF in the product mixture at any given time may be higher than those values given based on a single pass through the reactor. Such continuous processes are intended to fall within the scope of the invention, so long as the amount of 2,6-DCF formed per pass through the reactor, and attributable to reaction of the p-xylene fed to the reaction medium, is no more than those amounts already described.

In the processes according to the invention, the extent of solvent burned and rendered unusable, as estimated by carbon oxides formation, is the same as, or even reduced relative to, typical processes. While the absolute amount of carbon oxides formation may thus be reduced, this reduction is not achieved at the expense of acceptable conversion. Obtaining a low amount of carbon oxides formation may generally be achieved by running the reaction at low oxidation temperatures or using a catalyst which has a lower degree of conversion or selectivity, but this typically results in lowered conversion and increased quantities of intermediates. The processes of the invention have the advantage of maintaining a low ratio of solvent burn to conversion, thereby minimizing the impact on conversion to obtain the low solvent burn relative to other such processes.

Thus, in a preferred embodiment, the ratio of carbon oxides formation (in moles of CO and CO₂ expressed as COx, per mole of dialkyl aromatic feed), is no more than about 1.0 mole COₓ, or no more than about 0.5 mole COₓ, or no more than about 0.3 mole COₓ, in each case with respect to the molar quantity of dialkyl aromatic compounds fed to the reactor.

When production of methyl acetate is also taken into account, the total amount of CO, CO₂, and methyl acetate formed, expressed as moles per mole of dialkyl aromatic feed, is typically no more than about 1.2 moles, or no more than about 0.6 mole, or no more than about 0.3 mole, in each case per mole of dialkyl aromatic compounds fed to the reactor.

In an important aspect, the processes of the invention comprise reducing the amount of dicarboxyfluorenones produced. Thus, in various embodiments of the invention, the amount of 2,6-dicarboxyfluorenone produced, per batch or per pass through a reactor, may be no more than about 10 ppm, or no more than about 8 ppm, or no more than about 6 ppm, or from 0.4 to 10 ppm, or from 0.5 to 6 ppm, expressed with respect to the weight of the terephthalic acid produced.

In a further aspect, the amount of 2,6-dicarboxyfluorenone produced per batch or per pass through a reactor is no more than about 20 ppm, or no more than about 18 ppm, or no more than about 15 ppm, or from 1 to 20 ppm, or from 2 to 18 ppm, or from 3 ppm to 15 ppm, expressed with respect to the total weight of the reaction mixture.

Thus, in a process in accordance with the present invention, p-xylene is combined with, for example, a liquid comprising primarily acetic acid and water, in ratios to lead, for example, from about 2 to about 15 wt. % of p-xylene based on the weight of liquid feed entering the reactor, and an oxygen-containing gas, at a temperature from about 135° C. to about 165° C., using a catalyst composition comprising cobalt atoms and manganese atoms, with bromine atoms provided as a promoter, wherein the cobalt atoms are present in amounts as already described.

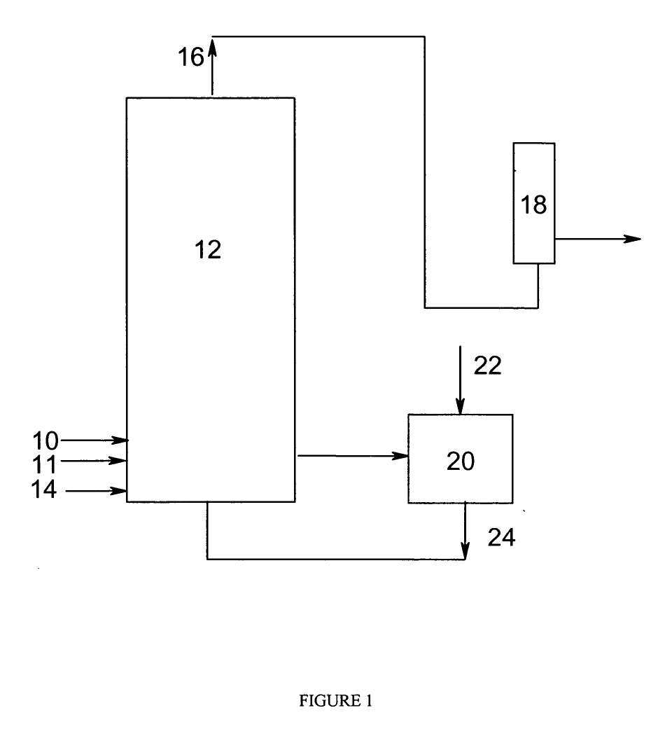

An embodiment of the invention will now be described referring to the accompanying FIG. 1, in which p-xylene is introduced via conduit 10 into primary oxidation reactor 12, and aqueous acetic acid solvent having dissolved therein the catalyst composition of the invention fed through line 11 to the reactor 12. If desired, the p-xylene, solvent, and catalyst composition charges may be fed to reactor 12 at a plurality of points, or fed together through one line. An oxygen-containing gas under pressure is introduced near the bottom of the reactor 12 via conduit 14. The preferred oxygen-containing gas is air or oxygen-enriched air. The flow rate of the oxygen-containing gas to reactor 12 is controlled to maintain between about 2 and 9 volume percent oxygen (calculated on a dry, solvent free basis) in the off-gas which exits the reactor via conduit 16. The reactants in reactor 12 are maintained at an elevated pressure of about 50 to 175 psia to maintain a contained, volatizable reaction medium substantially in the liquid state at the reaction temperature of about 135 to about 165° C.

During the course of the oxidation reaction, exothermic heat of reaction and water generated by the oxidation of p-xylene are removed from reactor 12 by vaporization of a portion of the liquid reaction medium. These vapors, known as reactor off-gas, comprise vaporized acetic acid solvent, about 5 to 30 weight percent water, and oxygen-depleted process gas containing minor amounts of decomposition products including catalyst residue, as well as additional carbon dioxide and carbon monoxide generated by the decomposition of acetic acid. The reactor off-gas passes upwardly through the reactor 12 and is conveyed via conduit 16 to the lower portion of water removal column 18 for distillation and recovery of the acetic acid back to the primary oxidation reactor. The crude reaction mixture is discharged from the primary oxidation reactor to a solid/liquid separator 20 into which is fed fresh acetic acid through line 22 to exchange the mother liquor discharged through line 24. The mother liquor containing acetic acid and the catalyst composition is subjected to conventional purification and purging techniques to recover and recycle the catalyst composition to the primary oxidation reactor 12.

Suitable dialkyl aromatic compounds useful as reactor feed-mixture components or ingredients in the methods of the present invention include dialkyl benzenes and naphthalenes such as o-xylene, m-xylene, p-xylene, 2,6-dimethylnaphthalene, 2,7-dimethylnaphthalene and 2,6-diisopropylnaphthalene. The respective aromatic carboxylic acid products of these alkyl aromatic compounds are orthophthalic acid, isophthalic acid, terephthalic acid (TPA), and 2,6- and 2,7-naphthalenedicarboxylic acids. The processes of the invention can be used to produce TPA and isophthalic acid, and are particularly well suited for the production of benzenedicarboxylic and naphthalenedicarboxylic acids, especially TPA.

Suitable aqueous aliphatic acid solvents useful in the methods of the invention are those that are readily volatilizable at the reaction temperatures. Among such solvents are aqueous solutions of C₂ to C₆ monocarboxylic acids, e.g., acetic acid, propionic acid, n-butyric acid, isobutyric acid, n-valeric acid, trimethylacetic acid, caprioic acid, and mixtures thereof. Preferably, the volatilizable monocarboxylic aliphatic acid solvent is an aqueous acetic acid solution.

As an example, p-xylene is oxidized to produce TPA practicing the method of the present invention, the usual process conditions and parameters being as already described. The contents of reactor 12 may be subjected to a pressure, for example, in the range of about 3.8 to about 7.9 bar absolute (about 55 to 115 psia) at a temperature in the range of 135° C. to 165° C.

Further description of the oxidation of alkyl aromatics to benzenepolycarboxylic acids may be found in the “Phthalic Acids and Other Benzenepolycarboxylic Acids” entry of Kirk-Othmer Encyclopedia of Chemical Technology, Vol 18, 4th ed., (1995) pp. 991-1043, the relevant portions of which are incorporated herein by reference.

The measure of toluic acid (p-TA) in the product mixture, being an incomplete oxidation product, is understood to indicate the degree of conversion achieved, with lower p-TA levels indicating higher conversion.

As described, the acetic acid solvent is decomposed, to some extent, in a side reaction to produce mainly carbon dioxide, carbon monoxide, and methyl acetate. The rate of acetic acid decomposition was estimated in the examples by measuring the number of moles of carbon dioxide and carbon monoxide exiting in the oxidizer vent gas, and in some of the examples, by determination of the moles of methyl acetate present in the oxidizer condensate. In the examples in which methyl acetate was measured, the off-gas is defined as the sum of the moles of carbon monoxide, carbon dioxide, and methyl acetate produced. To achieve satisfactory results for the oxidation process, the acetic acid decomposition should be low while the rate of xylene conversion to TPA is high (the concentration of toluic acid in the product mixture is low) and the concentration of DCF in the product is low. The amount of p-TA found in the oxidizer filtrate is a measure of the rate of the oxidation. The amount of DCF found in the solid product is a measure of quality. The amount of carbon oxides formation, at least in part the result of acetic acid decomposition, is a measure of cost of the oxidation process.

The invention has been described in detail with particular reference to preferred embodiments thereof, but it will be understood that variations and modifications can be effected within the spirit and scope of the invention. Moreover, all patents, patent applications (published or unpublished, foreign or domestic), literature references or other publications noted above are incorporated herein by reference for any disclosure pertinent to the practice of this invention.

### EXAMPLES

### Examples 1-30

In examples 1-30, oxidations of p-xylene to terephthalic acid were carried out in a pilot reactor system assembled around an agitated, hot-oil jacketed, 2-gallon, titanium reaction vessel. The gas dispersion type agitator within the reaction vessel can be rotated at various speeds. At about 1,500 revolutions per minute (rpm), the power draw of the agitator was approximately 210 watts. The pilot reactor system was equipped with means to control the pressure and temperature within the reaction vessel and to control the gas and liquid flow rates entering the reaction vessel.

para-Xylene was fed at an effectively steady rate of 2.64 moles per hour via a metering system. Catalyst feed solution was pumped from a catalyst feed tank into the reaction vessel at an effectively steady rate of 7.1 pounds per hour (3.2 kilogram per hour). Both para-xylene and catalyst feed solution were released into the reaction medium through a dip tube ending below the level of aerated slurry within the reaction vessel.

Using a nuclear level detection system, the reaction mass in the reactor was maintained at an indicated value of around 40 percent by operation of an automatic drain valve located near the bottom of the reactor. By separate calibration, this indicated level corresponded to approximately 3 kilograms of slurry mass within the aerated, agitated reaction vessel. Air was fed effectively continuously through a tube ending below the level of the gas dispersion impeller within the reaction vessel.

The off-gas from the reaction vessel was fitted with a condenser system designed to condense most organic vapor from the off-gas. Condensate from the off-gas was removed from the process at a rate of about 2.8 pounds per hour (1.3 kilogram per hour), and the balance of the off-gas condensate was returned to the reaction vessel. The air feed rate was adjusted to maintain an oxygen concentration in the exiting gas of about 3 to 4 mole percent on a dry basis after the off-gas condenser. The gases exiting the reactor were continuously monitored for oxygen, carbon dioxide, and carbon monoxide with in-line gas analyzers.

The product slurry, containing crude terephthalic acid (CTA) solids, was continuously collected in an unaerated, stirred receiving tank kept at a temperature from about 138° C. to about 140° C. This first tank was drained batch-wise every four hours into a second unaerated tank (about 40° C.). The resulting slurry was cooled as rapidly as possible after dropping it from the first tank. It was held in this second tank until the previous sample was removed from the filter. The slurry was then blown through the bag pressure filter, then nitrogen was used to blow most of the filtrate through the filter. The filtrate passing through the bag filter was drained, weighed and sampled without further processing. A sample of this filtrate was analyzed by liquid chromatography for compositional profile, by X-ray for metals, by gas chromatography for methyl acetate and xylene, and by near infrared for water.

Before the next scheduled draining from the first to the second tank, the nitrogen flow was stopped, the bag pressure filter was removed and the wet solids were collected into a sample bag. The wet solids were weighed for calculating a mass balance. A small sample of the wet solids was removed and placed in a moisture balance to measure the % moisture. About 100 g of the wet solids were collected and placed in a filtration funnel. They were washed at room temperature with acetic acid several times (until the acetic acid picked up no color). The washed solids were then removed from the filtration funnel and dried in a vacuum oven. A sample of these dried solids was analyzed by liquid chromatography.

The reaction condensate was weighed and analyzed by gas chromatography and near infrared.

For each oxidation run, the catalyst feed solution was prepared in an agitated catalyst feed tank. The catalyst feed solution contained glacial acetic acid and deionized water. The cobalt in the catalyst feed solution was added as cobaltous acetate tetrahydrate, the manganese was added as manganous acetate tetrahydrate, and the bromine was added as aqueous 48 percent hydrobromic acid. The amounts of each component in the catalyst feed solution were selected to give the reaction slurry compositions shown below.

For start-up, the reaction vessel was charged with catalyst feed solution and xylene, was brought to the reaction temperature with a heating oil jacket and pressure, and was concentrated by evaporation to about half volume. Air diluted with nitrogen was introduced into the mixture until an exotherm was observed. Once the reaction was initiated, para-xylene and catalyst feed solution were fed to the reaction at the rates given above. The reaction was sustained at the conditions for about 8 hours before product was retained. Thereafter, slurry product was collected at about 4-hour intervals for the duration of the experiment, and analyses were conducted as stated above. The values for the gas flows, temperature, and pressure were recorded every ten minutes and averaged for each run. The concentration of DCF in the solids listed in Tables 1-5 refers to the concentration of 2,6-DCF in the solid isolated using the procedure presented earlier. Likewise, the p-TA in the filtrate refers to the concentration of this reaction intermediate in the filtrate collected as described above. The concentrations of total 2,6-DCF refers to the total concentration of this impurity in the slurry exiting the oxidizer.

Table 1 lists results for oxidation runs carried out at 140-143° C. Table 2 lists results for runs at 148-153° C., and Table 3 lists results for 154-158° C. Table 4 contains results for runs at 159-161° C. Table 5 illustrates the relationship between temperature and acetic acid burn at similar conversions.

TABLE 1
Oxidations carried out at 140-143° C.

| | | | | | | | | DCF | | Total |
|---|---|---|---|---|---|---|---|---|---|---|
| | | | | | | | | (ppm) | p-TA | 2,6 |
| | T | P | water | [Co] | [Br] | [Mn] | mol | in | (ppm) | DCF |
| Example | (° C.) | (psig) | (%) | ppm | ppm | ppm | offgas/2 | solids | filtrate | (ppm) |
| 1 | 141 | 47 | 5.1 | 5935 | 4589 | 421 | 0.48 | 0.8 | 3216 | 0.48 |
| 2 | 140 | 47 | 5.6 | 2967 | 2352 | 202 | 0.63 | 1.8 | 4083 | 1.0 |
| 3 | 140 | 47 | 6.4 | 5091 | 4039 | 324 | 0.52 | 1.1 | 3183 | 0.57 |
| 4 | 141 | 48 | 6.1 | 4788 | 3614 | 131 | 0.69 | 0.9 | 1912 | 0.59 |
| 5 | 140 | 52 | 6.3 | 4066 | 3049 | 265 | 0.44 | 1.0 | 4283 | 0.68 |
| 6 | 140 | 47 | 6.4 | 2886 | 2362 | 193 | 0.60 | 1.9 | 4067 | 1.1 |
| 7 | 141 | 48 | 6.1 | 4795 | 3893 | 129 | 0.72 | 0.7 | 1927 | 0.52 |
| 8 | 141 | 46 | 6.1 | 3581 | 3338 | 57 | 0.83 | 1.1 | 1673 | 0.75 |
| 9 | 141 | 80 | 6.7 | 3795 | 3383 | 67 | 0.82 | 0.4 | 1699 | 0.38 |
| 10 | 141 | 50 | 5.8 | 3191 | 3444 | 136 | 0.75 | 0.8 | 1741 | 0.71 |
| 11 | 141 | 46 | 6.0 | 1633 | 2777 | 57 | 0.83 | 4.0 | 3861 | 2.1 |
| 12 | 140 | 46 | 5.7 | 1610 | 2754 | 289 | 0.71 | 3.9 | 5256 | 2.0 |
| 13 | 140 | 45 | 6.1 | 5303 | 3838 | 64 | 0.76 | 0.8 | 1605 | 0.71 |
| 14 | 140 | 46 | 5.8 | 1651 | 3642 | 294 | 0.96 | 2.8 | 3715 | 1.7 |
| 15 | 141 | 46 | 5.9 | 4734 | 3096 | 56 | 0.74 | 1.0 | 1591 | 0.67 |
| 16 | 141 | 46 | 5.8 | 3389 | 3456 | 175 | 0.66 | 1.3 | 2696 | 0.87 |
| 17 | 143 | 54 | 5.9 | 4890 | 3746 | 329 | 0.53 | 0.7 | 2962 | 0.56 |

Values in Table are averages for a 36-h run time.
mol offgas/2 = Sum of moles (CO + CO₂ + methyl acetate) averaged for 4-h time intervals taken over a 36-h run time divided by 2.

TABLE 2
Oxidations carried out at 148-152° C.

| | | | | | | | | | | Total |
|---|---|---|---|---|---|---|---|---|---|---|
| | | | | | | | | DCF | p-TA | 2,6 |
| | T | P | water | [Co] | [Br] | [Mn] | mol | (ppm) in | (ppm) | DCF |
| Ex. | (° C.) | (psig) | (%) | ppm | ppm | ppm | offgas/2 | solids | filtrate | (ppm) |
| 18 | 148 | 64 | 6.0 | 2470 | 1945 | 164 | 0.81 | 1.5 | 2743 | 0.98 |
| 19 | 148 | 67 | 6.4 | 4868 | 3610 | 330 | 0.71 | 0.6 | 2260 | 0.48 |
| 20 | 148 | 65 | 6.8 | 3783 | 2923 | 253 | 0.77 | 0.7 | 2268 | 0.55 |
| 21 | 152 | 74 | 5.7 | 2499 | 1932 | 168 | 0.77 | 1.1 | 2101 | 0.93 |
| 22 | 149 | 62 | 6.4 | 2476 | 2096 | 43 | 1.19 | 1.3 | 1191 | 1.2 |

Values in Table are averages for a 36-h run time.
mol offgas/2 = Sum of moles (CO + CO₂ + methyl acetate) averaged for 4-h time intervals taken over a 36-h run time divided by 2.

TABLE 3
Oxidations carried out at 155-157° C.

| | | | | | | | | | | Total |
|---|---|---|---|---|---|---|---|---|---|---|
| | | | | | | | | DCF | p-TA | 2,6 |
| | T | P | water | [Co] | [Br] | [Mn] | mol | (ppm) in | (ppm) | DCF |
| Ex. | (° C.) | (psig) | (%) | ppm | ppm | ppm | offgas/2 | solids | filtrate | (ppm) |
| 23 | 156 | 80 | 5.9 | 3796 | 2671 | 279 | 1.13 | 0.7 | 1551 | 0.86 |
| 24 | 155 | 80 | 5.8 | 1996 | 1580 | 140 | 1.09 | 1.8 | 1940 | 1.4 |
| 25 | 155 | 80 | 6.1 | 3068 | 2273 | 210 | 1.06 | 0.8 | 1567 | 0.75 |
| 26 | 156 | 78 | 5.9 | 3809 | 2768 | 270 | 1.05 | 0.7 | 1369 | 0.68 |
| 27 | 156 | 79 | 6.4 | 2061 | 1544 | 140 | 1.22 | 1.4 | 1625 | 1.1 |

Values in Table are averages for a 36-h run time.
mol offgas/2 = Sum of moles (CO + CO₂ + methyl acetate) averaged for 4-h time intervals taken over a 36-h run time divided by 2.

TABLE 4
Oxidations carried out at 160-161° C.

| | | | | | | | | DCF | | |
|---|---|---|---|---|---|---|---|---|---|---|
| | | | | | | | | (ppm) | p-TA | Total |
| | T | P | water | [Co] | [Br] | [Mn] | mol | in | (ppm) | 2,6 DCF |
| Ex. | (° C.) | (psig) | (%) | ppm | ppm | ppm | offgas/2 | solids | filtrate | (ppm) |
| 28 | 160 | 90 | 6.3 | 1775 | 1345 | 123 | 1.19 | 1.4 | 1598 | 1.2 |
| 29 | 161 | 90 | 5.9 | 1502 | 1697 | 66 | 1.45 | 1.4 | 1159 | 0.83 |
| 30 | 160 | 91 | 6.3 | 1716 | 1571 | 67 | 1.65 | 1.8 | 925 | 1.1 |

Values in Table are averages for a 36-h run time.
mol offgas/2 = Sum of moles (CO + CO₂ + methyl acetate) averaged for 4-h time intervals taken over a 36-h run time divided by 2.

TABLE 5
Comparison of examples at similar conversion and different
temperatures and catalyst concentrations.

| | | | | | | | | DCF | | Total |
|---|---|---|---|---|---|---|---|---|---|---|
| | | | | | | | | (ppm) | p-TA | 2,6 |
| | T | P | water | [Co] | [Br] | [Mn] | mol | in | (ppm) | DCF |
| Ex. | (° C.) | (psig) | (%) | ppm | ppm | ppm | offgas/2 | solids | filtrate | (ppm) |
| 13 | 140 | 45 | 6.1 | 5303 | 3838 | 64 | 0.76 | 0.8 | 1605 | 0.71 |
| 15 | 141 | 46 | 5.9 | 4734 | 3096 | 56 | 0.74 | 1.0 | 1591 | 0.67 |
| 22 | 149 | 62 | 6.4 | 2476 | 2096 | 43 | 1.19 | 1.3 | 1191 | 1.2 |
| 24 | 155 | 80 | 6.1 | 3068 | 2273 | 210 | 1.06 | 0.8 | 1567 | 0.75 |
| 27 | 156 | 79 | 6.4 | 2061 | 1544 | 140 | 1.22 | 1.4 | 1625 | 1.1 |
| 28 | 160 | 90 | 6.3 | 1775 | 1345 | 123 | 1.19 | 1.4 | 1598 | 1.2 |

Values in Table are averages for a 36-h run time.
mol offgas/2 = Sum of moles (CO + CO₂ + methyl acetate) averaged for 4-h time intervals taken over a 36-h run time divided by 2.

### Prophetic Examples 31-36

The data presented in Tables 1 through 4 was used to develop a theoretical polynomial model for each response reported in the tables. These models were then used to predict conditions leading to low levels of DCF in the solid with low values for mol offgas/2, as follows.

$$\begin{matrix}[ p - TA ] = 157164.4 - 2.34 [ Co ] + 1.50 [ Br ] + 10.13 [ Mn ] - 1878.66 ( T ) + \\ {( 2.86 \times {10}^{- 4} ) [ Co ]}^{2} - {( 4.33 \times {10}^{- 4} ) [ Br ]}^{2} + 5.70 {( T )}^{2} + \\ ( 1.71 \times {10}^{- 3} ) [ Co ] [ Mn ] - ( 2.41 \times {10}^{- 3} ) [ Br ] [ Mn ]\end{matrix}$$

$$\begin{matrix}[ DCF ] = 9.23 - ( 4.81 \times {10}^{- 3} ) [ Co ] + ( 4.70 \times {10}^{- 3} ) [ Br ] - 0.036 ( T ) + \\ {( 2.81 \times {10}^{- 7} ) [ Co ]}^{2} - {( 1.21 \times {10}^{- 6} ) [ Br ]}^{2} + ( 7.09 \times {10}^{- 7} ) [ Co ] [ Br ]\end{matrix}$$

$$\begin{matrix}[ Off gas / 2 ] = - 5.12 - ( 6.66 \times {10}^{- 5} ) [ Co ] + ( 2.34 \times {10}^{- 4} ) [ Br ] - \\ ( 1.88 \times {10}^{- 5} ) [ Mn ] + 0.0386 [ T ] - ( 2.33 \times {10}^{- 7} ) [ Co ] [ Mn ]\end{matrix}$$

Thus, Prophetic Examples 31-36 (Table 6) are carried out as set forth above with respect to Examples 1-30, the values in Table 6 being calculated values as just described.

TABLE 6
Prophetic Examples leading to low levels of DCF.

| | | | | | | | | | | DCF | p-TA |
|---|---|---|---|---|---|---|---|---|---|---|---|
| Prophetic | T | P | water | [Co] | [Br] | | [Mn] | | mol | (ppm) in | (ppm) |
| Example | (° C.) | (psig) | (%) | ppm | ppm | [Co]/[Br] | ppm | [Co]/[Mn] | offgas/2 | solids | filtrate |
| 31 | 140 | 45 | 6 | 4950 | 3580 | 1.4 | 100 | 50 | 0.67 | 1.1 | 2110 |
| 32 | 145 | 56 | 6 | 2800 | 1720 | 1.6 | 55 | 51 | 0.66 | 0.7 | 2180 |
| 33 | 144 | 54 | 6 | 5500 | 2800 | 2.0 | 65 | 85 | 0.64 | 0.7 | 2250 |
| 34 | 146 | 59 | 6 | 5950 | 2700 | 2.2 | 55 | 108 | 0.67 | 0.6 | 2240 |
| 35 | 140 | 45 | 6 | 5600 | 2950 | 1.9 | 135 | 41 | 0.42 | 1.1 | 4090 |
| 36 | 148 | 63 | 6 | 5950 | 2700 | 2.2 | 75 | 79 | 0.72 | 0.5 | 2110 |

### Examples 37-115

The oxidations of p-xylene described in examples 37 to 115 were carried out under conditions different from those used for Examples 1 through 30. Each reaction was performed in a 3-gal titanium agitated autoclave equipped with a means to control the pressure, temperature, gas flow, and a condenser system designed to remove a predetermined amount of condensed vapor from the process. Para-xylene was fed with a metering system at a rate of 330 g/h, and fresh catalyst solution was pumped from a feed tank into the autoclave at a rate of 3330 g/h. The gases exiting the reactor were continuously monitored for oxygen, carbon dioxide, and carbon monoxide with an in-line gas analyzer. The air feed rate was adjusted so as to maintain an oxygen concentration of that listed in the Tables. The level in the reactor was maintained at around 43% by operation of an automatic drain valve located in the bottom of the reactor. Reaction condensate was removed from the process at a rate of 2390 g/h. The product slurry was continuously collected in a receiving tank, which was drained batch-wise every four hours into a second tank in which the product was cooled to crystallize any dissolved product. The resulting slurry was filtered and the filtrate was collected and weighed and analyzed by liquid chromatography for low-level organic compounds, X-ray for metals, and % water was determined for the liquid portion by using a Karl Fisher titration procedure. A portion of the remaining solids was washed with acetic acid, dried and analyzed by liquid chromatography.

For each oxidation run, the catalyst feed mixture was prepared in the feed tank. The cobalt was added as cobalt hydroxide, the manganese was added as manganous acetate tetrahydrate, and the bromine was added as aqueous hydrobromic acid. The autoclave was charged with catalyst feed and xylene, brought to the reaction temperature and pressure, and was concentrated to about half volume. Air diluted with nitrogen was introduced into the mixture until an exotherm was observed. Once the reaction was initiated, xylene and catalyst were fed to the reaction at the rates given above. The reaction was sustained at the conditions for 8 h before product was retained. The reaction was continued and product was collected at 4-h intervals for a minimum of 20 h. The resulting data points were averaged to give average value for each run for water, organic products, and catalyst concentration. The values for the gas flows, temperature, and pressure were recorded every ten minutes and averaged for each run. The values for the COx/2 listed in these examples correspond to the total molar quantity of CO and CO₂ per mole of TPA produced, divided by 2. The concentrations of 2,6-DCF and total p-TA refer to the total concentration of these impurities in the slurry exiting the oxidizer.

TABLE 7
Oxidation of p-xylene

| | Co | Br | | Mn | | H2O | O2 | Pressure | Temp | COx/2 | 2,6 DCF | total p-TA |
|---|---|---|---|---|---|---|---|---|---|---|---|---|
| Examples | ppm | ppm | Co:Br | ppm | Co:Mn | % | % | psig | ° C. | mol/mol TPA | ppm | ppm |
| 37 | 4720 | 4500 | 1.0 | 236 | 20 | 4 | 3.0 | 70 | 152 | 0.07 | 4.0 | 1690 |
| 38 | 4720 | 2000 | 2.4 | 47 | 100 | 8 | 6.0 | 70 | 152 | 0.07 | 3.1 | 1900 |
| 39 | 4720 | 2000 | 2.4 | 47 | 100 | 4 | 3.0 | 70 | 152 | 0.08 | 4.5 | 1540 |
| 40 | 3304 | 3250 | 1.0 | 141.5 | 23 | 6 | 4.5 | 70 | 152 | 0.08 | 4.2 | 1620 |
| 41 | 1888 | 4500 | 0.4 | 236 | 8 | 4 | 6.0 | 70 | 152 | 0.09 | 4.4 | 2090 |
| 42 | 3304 | 3250 | 1.0 | 47 | 70 | 6 | 4.5 | 70 | 152 | 0.09 | 3.4 | 610 |
| 43 | 3304 | 4500 | 0.7 | 141.5 | 23 | 6 | 4.5 | 70 | 152 | 0.09 | 3.1 | 1240 |
| 44 | 1888 | 4500 | 0.4 | 236 | 8 | 8 | 3.0 | 70 | 152 | 0.08 | 11.4 | 2790 |
| 45 | 4720 | 2000 | 2.4 | 236 | 20 | 8 | 3.0 | 70 | 152 | 0.05 | 6.9 | 3050 |
| 46 | 1888 | 2000 | 0.9 | 47 | 40 | 4 | 6.0 | 70 | 152 | 0.06 | 6.6 | 2840 |
| 47 | 4720 | 3250 | 1.5 | 141.5 | 33 | 6 | 4.5 | 70 | 152 | 0.07 | 3.7 | 1750 |
| 48 | 1888 | 4500 | 0.4 | 47 | 40 | 8 | 6.0 | 70 | 152 | 0.09 | 5.9 | 2270 |
| 49 | 1888 | 2000 | 0.9 | 236 | 8 | 4 | 3.0 | 70 | 152 | 0.05 | 12.2 | 3650 |
| 50 | 3304 | 3250 | 1.0 | 141.5 | 23 | 6 | 6.0 | 70 | 152 | 0.08 | 3.9 | 1790 |
| 51 | 1888 | 4500 | 0.4 | 47 | 40 | 4 | 3.0 | 70 | 152 | 0.10 | 7.1 | 1810 |
| 52 | 1888 | 2000 | 0.9 | 236 | 8 | 8 | 6.0 | 70 | 152 | 0.05 | 12.1 | 5220 |
| 53 | 4720 | 4500 | 1.0 | 47 | 100 | 4 | 6.0 | 70 | 152 | 0.10 | 1.8 | 880 |
| 54 | 4720 | 4500 | 1.0 | 47 | 100 | 8 | 3.0 | 70 | 152 | 0.08 | 5.5 | 1590 |
| 55 | 3304 | 3250 | 1.0 | 141.5 | 23 | 6 | 4.5 | 70 | 152 | 0.08 | 3.7 | 1560 |
| 56 | 1888 | 2000 | 0.9 | 47 | 40 | 8 | 3.0 | 70 | 152 | 0.05 | 20.4 | 4780 |
| 57 | 1888 | 3250 | 0.6 | 141.5 | 13 | 6 | 4.5 | 70 | 152 | 0.08 | 5.1 | 2680 |
| 58 | 3304 | 3250 | 1.0 | 141.5 | 23 | 4 | 4.5 | 70 | 152 | 0.08 | 4.5 | 1660 |
| 59 | 3304 | 2000 | 1.7 | 141.5 | 23 | 6 | 4.5 | 70 | 152 | 0.06 | 4.7 | 2530 |
| 60 | 4720 | 2000 | 2.4 | 236 | 20 | 4 | 6.0 | 70 | 152 | 0.05 | 3.6 | 3080 |
| 61 | 3304 | 3250 | 1.0 | 141.5 | 23 | 8 | 4.5 | 70 | 152 | 0.06 | 8.6 | 2880 |
| 62 | 3304 | 3250 | 1.0 | 141.5 | 23 | 6 | 4.5 | 70 | 152 | 0.08 | 3.8 | 1570 |
| 63 | 3304 | 3250 | 1.0 | 236 | 14 | 6 | 4.5 | 70 | 152 | 0.07 | 4.6 | 2430 |
| 64 | 4720 | 4500 | 1.0 | 236 | 20 | 8 | 6.0 | 70 | 152 | 0.06 | 3.6 | 2090 |
| 65 | 3304 | 3250 | 1.0 | 141.5 | 23 | 6 | 3.0 | 70 | 152 | 0.08 | 5.5 | 1500 |
| 66 | 3304 | 3250 | 1.0 | 141.5 | 23 | 6 | 4.5 | 70 | 152 | 0.08 | 3.7 | 1700 |
| 67 | 2600 | 2600 | 1.0 | 200 | 13 | 4 | 3.0 | 90 | 160 | 0.10 | 5.1 | 1020 |
| 68 | 2600 | 2600 | 1.0 | 20 | 130 | 8 | 3.0 | 90 | 160 | 0.09 | 7.9 | 1610 |
| 69 | 1700 | 1700 | 1.0 | 110 | 15 | 6 | 4.5 | 90 | 160 | 0.09 | 5.8 | 1520 |
| 70 | 2600 | 800 | 3.3 | 200 | 13 | 8 | 3.0 | 90 | 160 | 0.05 | 13.7 | 4260 |
| 71 | 800 | 800 | 1.0 | 200 | 4 | 4 | 3.0 | 90 | 160 | 0.03 | 34.7 | 13670 |
| 72 | 2600 | 2600 | 1.0 | 200 | 13 | 8 | 6.0 | 90 | 160 | 0.09 | 3.9 | 1520 |
| 73 | 1700 | 1700 | 1.0 | 110 | 15 | 6 | 6.0 | 90 | 160 | 0.10 | 4.7 | 2760 |
| 74 | 1700 | 800 | 2.1 | 110 | 15 | 6 | 4.5 | 90 | 160 | 0.10 | 19.9 | 6980 |
| 75 | 1700 | 1700 | 1.0 | 110 | 15 | 6 | 4.5 | 90 | 160 | 0.09 | 8.0 | 2420 |
| 76 | 800 | 800 | 1.0 | 20 | 40 | 4 | 6.0 | 90 | 160 | 0.04 | 17.0 | 16390 |
| 77 | 2600 | 800 | 3.3 | 200 | 13 | 4 | 6.0 | 90 | 160 | 0.07 | 5.8 | 4060 |
| 78 | 800 | 2600 | 0.3 | 20 | 40 | 4 | 3.0 | 90 | 160 | 0.04 | 42.1 | 13330 |
| 79 | 800 | 2600 | 0.3 | 200 | 4 | 4 | 6.0 | 90 | 160 | 0.09 | 5.7 | 2330 |
| 80 | 2600 | 1700 | 1.5 | 110 | 24 | 6 | 4.5 | 90 | 160 | 0.10 | 4.8 | 2480 |
| 81 | 1700 | 1700 | 1.0 | 20 | 85 | 6 | 4.5 | 90 | 160 | 0.11 | 5.6 | 2310 |
| 82 | 800 | 2600 | 0.3 | 200 | 4 | 8 | 3.0 | 90 | 160 | 0.09 | 30.4 | 6160 |
| 83 | 1700 | 2600 | 0.7 | 110 | 15 | 6 | 4.5 | 90 | 160 | 0.11 | 7.2 | 1760 |
| 84 | 1700 | 1700 | 1.0 | 110 | 15 | 6 | 4.5 | 90 | 160 | 0.09 | 8.9 | 2210 |
| 85 | 800 | 1700 | 0.5 | 110 | 7 | 6 | 4.5 | 90 | 160 | 0.05 | 25.2 | 6520 |
| 86 | 1700 | 1700 | 1.0 | 110 | 15 | 6 | 4.5 | 90 | 160 | 0.10 | 7.1 | 1810 |
| 87 | 2600 | 800 | 3.3 | 20 | 130 | 8 | 6.0 | 90 | 160 | 0.07 | 6.9 | 2500 |
| 88 | 1700 | 1700 | 1.0 | 200 | 9 | 6 | 4.5 | 90 | 160 | 0.08 | 8.1 | 2500 |
| 89 | 800 | 800 | 1.0 | 200 | 4 | 8 | 6.0 | 90 | 160 | 0.04 | 26.8 | 10810 |
| 90 | 1700 | 1700 | 1.0 | 110 | 15 | 6 | 3.0 | 90 | 160 | 0.09 | 10.5 | 1990 |
| 91 | 1700 | 1700 | 1.0 | 110 | 15 | 8 | 4.5 | 90 | 160 | 0.08 | 9.5 | 2510 |
| 92 | 800 | 800 | 1.0 | 20 | 40 | 8 | 3.0 | 90 | 160 | 0.03 | 45.1 | 15520 |
| 93 | 1700 | 1700 | 1.0 | 110 | 15 | 4 | 4.5 | 90 | 160 | 0.09 | 6.6 | 1840 |
| 94 | 2600 | 800 | 3.3 | 20 | 130 | 4 | 3.0 | 90 | 160 | 0.09 | 9.3 | 3110 |
| 95 | 800 | 2600 | 0.3 | 20 | 40 | 8 | 6.0 | 90 | 160 | 0.06 | 30.3 | 11870 |
| 96 | 2600 | 2600 | 1.0 | 20 | 130 | 4 | 6.0 | 90 | 160 | 0.16 | 2.5 | 900 |
| 97 | 1600 | 1600 | 1.0 | 220 | 7 | 8 | 4.5 | 110 | 170 | 0.12 | 7.7 | 1450 |
| 98 | 600 | 600 | 1.0 | 40 | 15 | 8 | 3.0 | 110 | 170 | 0.05 | 78.5 | 22230 |
| 99 | 600 | 600 | 1.0 | 400 | 2 | 8 | 6.0 | 110 | 170 | 0.05 | 38.9 | 13600 |
| 100 | 2600 | 2600 | 1.0 | 40 | 65 | 8 | 3.0 | 110 | 170 | 0.14 | 9.2 | 990 |
| 101 | 2600 | 2600 | 1.0 | 400 | 7 | 8 | 6.0 | 110 | 170 | 0.10 | 6.8 | 1880 |
| 102 | 2600 | 600 | 4.3 | 40 | 65 | 8 | 6.0 | 110 | 170 | 0.14 | 6.2 | 2090 |
| 103 | 2600 | 600 | 4.3 | 400 | 7 | 8 | 3.0 | 110 | 170 | 0.08 | 15.1 | 3820 |
| 104 | 600 | 2600 | 0.2 | 400 | 2 | 8 | 3.0 | 110 | 170 | 0.16 | 18.4 | 4200 |
| 105 | 3800 | 1300 | 2.9 | 110 | 35 | 6 | 4.5 | 90 | 160 | 0.12 | 3.8 | 1590 |
| 106 | 4800 | 1300 | 3.7 | 110 | 44 | 6 | 4.5 | 90 | 160 | 0.12 | 4.4 | 1870 |
| 107 | 2500 | 2600 | 1.0 | 200 | 13 | 6 | 4.5 | 80 | 156 | 0.08 | 5.8 | 2180 |
| 108 | 1700 | 1800 | 0.9 | 110 | 15 | 6 | 4.5 | 80 | 156 | 0.06 | 12.9 | 4700 |
| 109 | 3700 | 1800 | 2.1 | 110 | 34 | 6 | 4.5 | 80 | 156 | 0.07 | 6.0 | 2630 |
| 110 | 400 | 1000 | 0.4 | 1000 | 0 | 6 | 4.5 | 170 | 185 | 0.12 | 9.5 | 3500 |
| 111 | 1970 | 1740 | 1.1 | 118 | 17 | 6 | 3.0 | 90.0 | 160 | 0.09 | 10.6 | 2340 |

### Examples 112-115

Examples 112-115 (Table 8), conducted as in Examples 37-111 above, further demonstrate that decreasing the temperature alone is not sufficient to decrease the generation rate of 2,6-DCF if the Co:Mn ratio remains low at a value of 17.

TABLE 8
Oxidations carried out at different temperatures and same catalyst
concentrations

| | | | | | | | | 2,6- | total |
|---|---|---|---|---|---|---|---|---|---|
| | | P | water | [Co] | [Br] | [Mn] | COx/2 mol/mol | DCF | p-TA |
| Entry | T (° C.) | (psig) | (%) | ppm | ppm | ppm | TPA | (ppm) | (ppm) |
| 112 | 157 | 90 | 6.5 | 3058 | 2784 | 187 | 0.07 | 8.9 | 955 |
| 113 | 150 | 80 | 6.5 | 3058 | 2784 | 187 | 0.05 | 14.2 | 1740 |
| 114 | 145 | 60 | 6.5 | 3058 | 2784 | 187 | 0.04 | 14.7 | 2770 |
| 115 | 160 | 90 | 6.5 | 3058 | 2784 | 187 | 0.07 | 12.8 | 1239 |

### Examples 116-119

Examples 116-119 were carried out as above in Examples 37-111, and the data set forth in Table 9 below.

TABLE 9
Oxidation of p-xylene

| | Co | Br | | Mn | | H2O | O2 | Pressure | Temp | COx/2 | 2,6 DCF | total p-TA |
|---|---|---|---|---|---|---|---|---|---|---|---|---|
| Examples | ppm | ppm | Co:Br | ppm | Co:Mn | % | % | psig | ° C. | mol/mol TPA | ppm | ppm |
| 116 | 2470 | 950 | 2.6 | 25 | 99 | 5 | 3 | 90 | 160 | 0.10 | 6.8 | 1520 |
| 117 | 4520 | 1230 | 3.7 | 47 | 96 | 5 | 3 | 90 | 160 | 0.11 | 5.5 | 1520 |
| 118 | 4410 | 2250 | 2.0 | 32 | 138 | 8 | 3 | 75 | 154 | 0.10 | 4.8 | 1210 |
| 119 | 4770 | 3150 | 1.5 | 31 | 154 | 8 | 3 | 70 | 152 | 0.08 | 6.0 | 1830 |

### Prophetic Examples 120-126

Theoretical polynomial models were developed for the COx/2,2,6-DCF and total p-TA using the data only from Examples 37-96. These models were used to predict the conditions leading to a decrease in the generation rate of DCF while keeping the mole of COx divided by 2 per mole of TPA produced to a low value. These numerical models are as set forth below.

$$\begin{matrix}total p - TA ( ppm ) = exp ( 23.95391 - 1.673 \times {10}^{- 3} [ Co ] - \\ 8.740 \times {10}^{- 4} [ Br ] - 3.384 \times {10}^{- 3} [ Mn ] + \\ 0.0643 [ {H}_{ 2} O ] - 0.0800 [ Temp ] + 1.9376 \times \\ {{10}^{- 7} [ Co ]}^{2} + 1.027 \times {{10}^{- 7} [ Br ]}^{2} + \\ 1.607 \times {10}^{- 6} [ Co ] [ Mn ] )\end{matrix}$$

$$\begin{matrix}2 , 6 - DCF ( ppm ) = 1 / ( 1.1253 - 9.412 \times {10}^{- 4} [ Co ] + 4.008 \times {10}^{- 5} [ Br ] + \\ 3.059 \times {10}^{- 4} [ Mn ] - 0.0200 [ {H}_{ 2} O ] + 0.0397 [ {O}_{2} ] - \\ 7.195 \times {10}^{- 3} [ Temp ] - 1.585 \times {10}^{- 7} [ Co ] [ Mn ] + \\ { 6.724 \times {10}^{- 6} [ Co ] [ Temp ] )}^{2}\end{matrix}$$

$$\begin{matrix}COx / 2 = ( - 1.520 + 9.095 \times {10}^{- 5} [ Co ] + 4.424 \times {10}^{- 5} [ Br ] + \\ 2.133 \times {10}^{- 4} [ Mn ] + 0.0491 [ {H}_{2} O ] + 9.320 \times {10}^{- 3} [ Temp ] - \\ 7.881 \times {{10}^{- 9} [ Co ]}^{2} - 4.434 \times {{10}^{- 3} [ {H}_{2} O ]}^{2} - \\ { 6.995 \times {10}^{- 9} [ Co ] [ Br ] - 1.164 \times {10}^{- 7} [ Co ] [ Mn ] )}^{2}\end{matrix}$$

Thus, Prophetic Examples 120 to 126 (Table 10) are carried out as set forth above with respect to Examples 37-115, the values in Table 10 being calculated values.

TABLE 10
Prophetic examples for the oxidation of p-xylene leading to low level of DCF.

| Prophetic | Co | Br | | Mn | | H2O | O2 | Pressure | Temp | COx/2 | 2,6 DCF | total p-TA |
|---|---|---|---|---|---|---|---|---|---|---|---|---|
| Examples | ppm | ppm | Co:Br | ppm | Co:Mn | % | % | psig | ° C. | mol/mol TPA | ppm | ppm |
| 120 | 2800 | 1200 | 2.3 | 20 | 140 | 8 | 3 | 90 | 160 | 0.09 | 7.9 | 2050 |
| 121 | 3400 | 1500 | 2.3 | 38 | 90 | 6 | 3 | 90 | 160 | 0.12 | 4.3 | 1200 |
| 122 | 2500 | 3800 | 0.7 | 20 | 125 | 5 | 3 | 77 | 155 | 0.11 | 5.4 | 1190 |
| 123 | 3740 | 4500 | 0.8 | 45 | 85 | 8 | 3 | 70 | 152 | 0.09 | 4.7 | 1130 |
| 124 | 3900 | 4500 | 0.9 | 85 | 46 | 8 | 3 | 70 | 152 | 0.09 | 4.7 | 1240 |
| 125 | 3000 | 3800 | 0.8 | 20 | 150 | 8 | 3 | 70 | 152 | 0.09 | 6.8 | 1370 |
| 126 | 4500 | 4390 | 1.0 | 41 | 111 | 8 | 3 | 70 | 152 | 0.09 | 3.8 | 1100 |

The many features and advantages of the invention are apparent from the detailed specification and, thus, it is intended by the appended claims to cover all such features and advantages of the invention which fall within the true spirit and scope of the invention. Further, since numerous modifications and changes will readily occur to those skilled in the art, it is not desired to limit the invention to the exact construction and operation illustrated and described, and accordingly all suitable modifications and equivalents may be resorted to, falling within the scope of the invention.

We claim:

1. A process for producing terephthalic acid, the process comprising combining in a reaction medium p-xylene, a solvent comprising water and a saturated organic acid having from 2-4 carbon atoms, and an oxygen-containing gas, at a temperature from about 135° C. to about 165° C., in the presence of a catalyst composition comprising cobalt, manganese, and bromine, wherein the amount of cobalt is from about 1,800 ppm to about 6,000 ppm, with respect to the total weight of the liquid in the reaction medium, and the weight ratio of cobalt to manganese is from about 40 to about 400.

2. The process according to claim 1, wherein the weight ratio of cobalt to manganese in the reaction mixture is from about 45 to about 250.

3. The process according to claim 1, wherein the weight ratio of cobalt to manganese in the reaction mixture is from about 50 to about 250.

4. The process according to claim 1, wherein the saturated organic acid comprises acetic acid.

5. The process according to claim 1, wherein the temperature is from about 140° C. to about 165° C.

6. The process according to claim 1, wherein the temperature is from 140° C. to 160° C.

7. The process according to claim 1, wherein the cobalt is present in an amount from about 1,900 ppm to about 6,000 ppm, with respect to the weight of the liquid in the reaction medium.

8. The process according to claim 1, wherein the cobalt is present in an amount from about 2,000 ppm to about 6,000 ppm, with respect to the weight of the liquid in the reaction medium.

9. The process according to claim 1, wherein the cobalt is present in an amount from about 2,500 ppm to about 5,000 ppm, with respect to the weight of the liquid in the reaction medium.

10. The process according to claim 1, wherein the amount of 2,6-dicarboxyfluorenone produced is no greater than about 10 ppm, with respect to the weight of the terephthalic acid produced.

11. The process according to claim 1, wherein the amount of 2,6-dicarboxyfluorenone produced is no greater than about 8 ppm, with respect to the weight of the terephthalic acid produced.

12. The process according to claim 1, wherein the amount of 2,6-dicarboxyfluorenone produced is no greater than about 20 ppm, with respect to the total weight of the reaction medium.

13. The process according to claim 1, wherein the amount of 2,6-dicarboxyfluorenone produced is no greater than about 18 ppm, with respect to the total weight of the reaction medium.

14. The process according to claim 1, wherein the amount of 2,6-dicarboxyfluorenone produced is from about 0.4 ppm to about 10 ppm, with respect to the weight of the terephthalic acid produced.

15. The process according to claim 1, wherein the total amount of COₓ produced is no greater than about 1.0 mole per mole of p-xylene provided to the reaction medium.

16. The process according to claim 1, wherein the total amount of COₓ produced is no greater than about 0.3 mole per mole of p-xylene provided to the reaction medium.

17. The process according to claim 1, wherein the total moles of carbon monoxide, carbon dioxide, and methyl acetate produced is no greater than about 1.2 moles per mole of p-xylene provided to the reaction medium.

18. The process according to claim 1, wherein the total moles of carbon monoxide, carbon dioxide, and methyl acetate produced is no greater than about 0.3 mole per mole of p-xylene provided to the reaction medium.

19. The process according to claim 1, wherein the cobalt is provided as one or more of: cobalt bromide, cobalt nitrate, cobalt chloride, cobalt acetate, cobalt octanoate, cobalt benzoate, or cobalt naphthalate.

20. The process according to claim 1, wherein the oxygen-containing gas comprises air.

21. The process according to claim 1, wherein the manganese is provided in an amount from about 20 ppm to about 425 ppm, with respect to the weight of liquid in the reaction medium.

22. The process according to claim 1, wherein the manganese is provided in an amount from about 20 ppm to about 300 ppm, with respect to the weight of liquid in the reaction medium.

23. The process according to claim 1, wherein the manganese is provided in an amount from about 20 ppm to about 200 ppm, with respect to the weight of liquid in the reaction medium.

24. The process according to claim 1, wherein the bromine is provided in an amount from about 800 ppm to about 4,600 ppm, with respect to the weight of liquid in the reaction medium.

25. The process according to claim 1, wherein the bromine is provided in an amount from about 1,200 ppm to about 4,200 ppm, with respect to the weight of liquid in the reaction medium.

26. The process according to claim 1, wherein the weight ratio of cobalt to bromine is from about 0.7 to about 3.5.

27. The process according to claim 1, wherein the weight ratio of cobalt to bromine is from about 0.8 to about 3.0.

28. The process according to claim 1, wherein the weight ratio of cobalt to bromine is from about 1.0 to about 2.5.

29. The process according to claim 1, wherein sodium is provided to the reaction medium in an amount from about 10 ppm to about 1,500 ppm, with respect to the weight of liquid in the reaction medium.

30. The process according to claim 1, wherein sodium is provided to the reaction medium in an amount from about 100 ppm to about 1,000 ppm, with respect to the weight of liquid in the reaction medium.
